(19) United States
(12) Patent Application Publication
(10) Pub. No.: US 2022/0202918 A1
(43) Pub. Date: Jun. 30, 2022

(54) METHODS OF TREATING MUSCULOSKELETAL DEFORMITIES IN QUADRUPED ANIMALS

(71) Applicant: VETMED THERAPEUTICS, INC., Gulf Breeze, FL (US)

(72) Inventors: J. Ben Renfroe, Gulf Breeze, FL (US); Daniel W. Carter, Gulf Breeze, FL (US)

(21) Appl. No.: 17/612,089

(22) PCT Filed: May 15, 2020
(86) PCT No.: PCT/US2020/033061

§ 371 Date: Nov. 17, 2021

Related U.S. Application Data
(60) Provisional application No. 62/849,316, filed on May 17, 2019.

(51) Int. Cl. A61K 38/48; A61K 9/00; A61P 21/00

(52) U.S. Cl. CPC A61K 38/4893; A61P 21/00; A61K 9/0019

(57) ABSTRACT

Methods of treating musculoskeletal deformities of an anatomical structure in a quadruped animal with botulinum toxin are described. Such deformities can be rotational or flexural. Such deformities can be acquired or congenital. Such anatomical structures include a limb, neck, torso, and/or pelvis of the quadruped animal.

### FIELD OF THE INVENTION

The invention relates to the treatment of musculoskeletal abnormalities in quadruped animals with botulinum toxin. More particularly, the method is effective in treating musculoskeletal abnormalities in the limb of a quadruped animal, such as hooved animals and other quadrupeds that are less than 3 years old.

### BACKGROUND OF THE INVENTION

Limb abnormalities of the foal are a common malady that markedly decreases the monetary value and functional use of an animal, such as a horse. These conditions can be present at birth or may develop shortly thereafter. These conditions are generally divided into three categories, namely, flexural, angular and rotational dysfunction, noting that some conditions can be more than one type. Particularly severe deformities if uncorrected can lead to lameness and even euthanasia.

Flexural abnormalities refer to hyperextension (e.g., “back-at-the-knee” and “dropped fetlock”) and hyperflexion (the inability to fully extend the coffin, pastern, fetlock and carpal joints). Current interventions for flexural anomalies include medical management, surgical intervention and farrier care. Medical management attempts to control factors contributing to the feedback cycle of pain and contraction, balanced nutrition to normalize bone and joint development, and judicious use of anti-inflammatory medication to reduce pain are the most common interventions. Modification of exercise is used to reduce concussion of growing bone and cartilage, and to increase stretching for tendon abnormalities. Adjunctive therapy often includes hoof trimming and/or shoes to alter the heel angle, medial to lateral hoof balance, reduction of the effective tensile forces on the involved muscles, altering the load of the boney growth plates (physis), and/or reduction of pain. Surgical intervention may be utilized for cases not responding to more conservative therapy. For example, cases of superficial digital flexor contraction that do not show significant improvement in four weeks may electively undergo desmotomy of the accessory ligament of the deep digital flexor tendon, i.e., the inferior check ligament, as a last resort. Desmotomy typically results in a mild functional lengthening of the superficial digital flexor tendon and reduced tensile forces on flexion. Reports indicate that horses subjected to the procedure are able to perform, but data is limited regarding the degree of post-surgical function and the long-term effects.

Angular deformities (crooked legs) describe deviation of a limb originating at a limb joint. Current theory suggests the animals that experience perinatal factors such as prematurity, placentitis, perinatal soft tissue trauma, laxity of soft tissues or acquired developmental factors affecting bone production and deposition across a physis such as nutrition, periarticular laxity, compressive injury, infection or restriction of long bone growth by the supportive connective tissue periosteum. The deformity is termed valgus—distal bone deviates outward and varus—distal bone deviates inward. For example, the most common deviation seen in young foals is in one or both fore limbs deviating outward from the knee joint: carpus valgus. Common medical treatment includes stall rest, rigid splints and/or casting the affected limb, corrective hoof trimming with shoes and extensions. Surgical treatment is considered when foals do not appear to respond to medical treatment and in foals that appear to have experienced a rapid growth rate and includes periosteal elevation, transphyseal bridging with screws and/or wires, osteotomy and ostectomy. The current treatments do not address the effect of the myotatic reflex which determines the vertical loading of the limb and its component joints, growth plates and bones. The vector forces of weight bearing during protraction, stance, and retraction are modified by ligament and muscle tone.

Rotational deformities refer to internal or external rotation of the foot in the sagittal plane (e.g., toe in or toe out). The forelimb is attached to the relatively laterally compressed thorax in quadrupeds by muscular and ligamentous attachments to form a sling-like structure (synsarcosis). Muscular tone of the component muscle bellies may alter the posture of the limb and its load bearing vector forces relative to the sagittal plane of the body. For example: Increased muscular tone of the cranial shoulder and pectoral muscles may result in rotation of the limb inward (pigeon toe) and increase the adduction of the limb (base-narrow); whereas increased tone of the caudal shoulder muscles may result in outward limb rotation (splay foot) and abduction of the limb (base-wide). Intervention for rotational anomalies is poorly substantiated by research and currently includes foot trimming, shoes with extensions and orthopedic surgical intervention.

Accordingly, there is a need for a proactive treatment that effectively treats or prevents the limb abnormalities of a foal, particularly one that might avoid the need for surgery and its associated risks. The present invention addresses such a need.

### SUMMARY OF THE INVENTION

The present invention relates to methods of treating musculoskeletal abnormalities, particularly those that stem from an imbalance in muscle tone in specific muscle groups of the leg and shoulder, in young hooved animals and domesticated quadrupeds in need thereof by administering a botulinum toxin composition to one or more muscles. In another of its aspects, the present invention provides a method of reducing the severity of flexural, angular or rotational abnormalities or the debilitating effects thereof. In an embodiment, flexural, angular or rotational limb abnormalities are treated by injecting a botulinum toxin composition to one or more muscles of the leg or shoulder. Methods described herein can be used to effectively treat rotational angular and flexural abnormalities, including those that might otherwise require surgery.

Embodiments of the present disclosure can include a method for treating asymmetric muscle tone in a limb or a rotational abnormality in a limb of a quadruped animal comprising: injecting a Botulinum Toxin, in a therapeutically effective amount, into one or more muscles of the limb or shoulder of the animal. The Botulinum Toxin can be injected into at least one muscle that imparts torsional forces on one or more skeletal bones of the limb. Botulinum Toxin can be injected into at least one muscle that exerts torsional forces on one or more skeletal bones of the torso, pelvis, or neck. Such muscles can include one or more muscles of the limb or shoulder are selected from lateral digital extensor, the common digital extensor, extensor carpi radialis, abductgor digiti longus, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi.

Another embodiment of the present disclosure includes a method for treating asymmetric or abnormal muscle tone or a musculoskeletal deformity of a quadruped animal comprising: injecting a Botulinum Toxin, in a therapeutically effective amount, into at least one muscle that exerts torsional forces on one or more skeletal bones of the anatomical structure. The one or more muscles that impart rotational forces on an anatomical structure can be selected from lateral digital extensor, the common digital extensor, extensor carpi radialis, abductgor digiti longus, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi.

Yet another embodiment of the present disclosure includes a method of treating asymmetric or abnormal muscle tone in a limb or a musculoskeletal deformity in a limb of a quadruped animal comprising: injecting a Botulinum Toxin, in a therapeutically effective amount, into (1) a superficial or deep digital flexor muscle of the distal limb and (2) one or more muscles selected from a common digital extensor, a right lateral digital extensor, ulnaris lateralis, flexor carpi ulnaris, lacertis fibrosis, flexor carpi radialis, extensor carpi radialis, biceps brachii, triceps, serratus ventralis, serratus cervicis, serratus thoracis, infraspinatus, brachiocephalicus, omotransversarius, trapezius, rhomboideus, cutaneous trunci, ascending pectoral muscles, descending pectoral muscles, transverse pectoral muscles, and latissimus dorsi.

Other embodiments of the present disclosure include a method of treating assymetric muscle tone in a limb or a deformity of a limb of a quadruped animal comprising: injecting a Botulinum Toxin, in a therapeutically effective amount, into one or more muscles of the distal limb selected from lateral digital extensor, the common digital extensor, ulnaris lateralis, flexor carpi ulnaris, lacertis fibrosis, flexor carpi radialis, extensor carpi radialis, biceps brachii, triceps, serratus ventralis, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi.

Animals to be treated by the methods described herein can be a horse and particularly a filly or a colt or a horse less than 3 years old or less than a year old.

The musculoskeletal deformity to be treated by the methods described herein can be a rotational or flexural deformity that is acquired or congenital.

### DETAILED DESCRIPTION OF THE INVENTION

The methods of the invention are directed to treating or reducing the severity of limb deformities in quadrupeds, particularly those that are hooved or domesticated. Limb deformities are a common congenital or acquired malady affecting quadrupeds, particularly larger, long-legged animals, such as, without limitation, horses, zebras, giraffes, and camels. Asymmetric tone in muscle(s) may be an underlying contributing factor to the rotational or flexural limb deformities.

While not wishing to be bound by a particular theory, abnormal and unbalanced muscle tone in specific muscle groups of the leg and shoulder may contribute to musculoskeletal deformities, such as flexural or rotational deformities. As such, intervention at the muscular and myoneural level through the administration of botulinum toxin can result in an improvement or stabilization of such deformities.

Flexural deformities are divided into congenital and acquired types. Congenital deformities are present at birth, and acquired deformities develop at some stage of the growing period. Most cases of acquired flexural deformities occur between the ages of six weeks and six months of age.

Abnormal and unbalanced muscle tone in specific muscle groups of the leg and shoulder may contribute to these deformities. Excessive tensile forces on the deep digital flexor tendon (DDFT) and/or the superficial digital flexor tendon (SDFT) can be a factor. However, excessive tensile forces on other muscle groups can also impact these deformities, particularly those of the dorsal shoulder girdle or synsarcosis (e.g., brachiocephalicus, omotransversarius, trapezius, rhomboideus, cutaneous trunci, latissimus dorsi, triceps, serratus ventralis, serratus cervicis, serratus thoracis, infraspinatus, and ascending, descending, and transverse pectorals), the “stay apparatus” (biceps brachii, superficial digital flexor (SDF), deep digital flexor (DDF), common digital extensor, extrensor carpi radialis, lacertus fibrosis), and contributors to the stay apparatus (e.g., ulnaris lateralis, flexor carpi ulnaris, and/flexor carpi radialis).

Flexural deformities include contracture of the DDFT, contracture of the SDFT, lateral digital flexor, flexor carpi ulnaris and radialis which are the direct effectors of flexural disorders secondary to injury, pain of Developmental Orthopedic Disease (DOD) and its related group of disorders including disorders of bone growth. Extensor deformities could result from excess limb extensor muscle tone secondary to injury to the brain, spinal cord, peripheral nerves or muscle belly. Potential effectors of forelimb extensor deformities include ulnaris lateralis, common digital extensor and extensor carpi radialis. Although research is not available regarding more proximal muscle involvement in flexor and extensor deformities, muscles of the elbow and shoulder exert effect upon flexural and extensor deformity. The biceps brachii has a direct effect through the fibrous tendon of the lacertus fibrosus of the “stay apparatus”. The coracobrachialis, triceps, teres major, teres minor and tensor fascia antebrachii influence deformity of the lower limb through affecting range of motion of the proximal limb via the tendon and muscular actions of the stay apparatus.

Contractures of the DDFT or SDFT are acquired flexor conditions that can affect any equine limb but most commonly affects one or both forelimbs. Imbalance, asymmetry, or dystonia of the muscles of the forearm and shoulder of the animal may result in remodeling of adjacent soft tissues and bone. These changes result in contracture and permanent flexural abnormalities of form and function in the affected limb, limiting the use and economic value of the animal.

For example, contracture of the DDFT can manifest through disuse of a limb, such as through an injury. The DDFT is attached to the solar aspect of the coffin bone and its muscle bellies (flexor digitorum profundus). Its function is to flex the limb during movement. When an injured limb is in disuse, the flexor muscle flexes the distal interphalangeal joint (coffin joint), predisposing it to raise the heel off the ground and bear weight on the toe. The affected hoof starts to become more cylindrical in shape from lack of normal weight distribution and contact with the ground. As the case advances, the angle of the dorsal hoof wall increases, approaching 90 degrees; the heels do not wear; the toe wears excessively; and bruising may be evident at the toe. At this stage, a pain/contraction cycle will continue to force the foal to place less weight on the affected limb while discomfort caused by the stretch of the contracted muscle/tendon results in a short, stiff gait. Interosseous and pericapsular soft tissue may then remodel to accommodate the abnormal position. Over time the distal border of the coffin bone may have radiographic evidence of bone lysis and resorption and more proximal limb muscles (biceps brachii, triceps and serratus ventralis) maintain the stay apparatus in an excessive flexed position. Muscles targeted for botulinum intervention to treat contracture of the DDFT can include one or more of the deep digital flexor, the biceps brachii, triceps, and the serratus ventralis. In some embodiments, muscles targeted for botulinum intervention to treat DDFT contracture comprises the deep digital flexor and one or more of the biceps brachii, triceps, and the serratus ventralix.

Contraction of the SDFT is a flexural deformity that can result in a dorsal movement of the inter-phalangeal joint and an increased dorsal angle of the pastern region. The underlying myotactic reflex is likely altered effectively decreasing the range of motion of the inter-phalangeal joint. In this disorder, the superficial digital flexor (SDF) appears to be the sole effector and therefore would be the muscle targeted for botulinum intervention to treat contracture of the SDFT.

Acquired flexural deformities can also result from developmental orthopedic deformities (DOD). DOD is shorthand for a group of orthopedic diseases of foals believed to result from influences of environment, nutritional imbalances, and/or genetic predisposition. Natal and pre-natal insult or injury may also be involved. DODs include osteochondritis dissecans, subchondral cystic lesions, physitis, angular and rotational deformities, cubodial bone abnormalities, and juvenile osteoarthritis. In some cases of DOD, flexural deformities are secondary to osteochondrosis, cystic lesions or physitis. Regardless, the resulting dysfunction of limb use may lead to alteration in muscle tone by altering the myotactic reflex. The target muscles for acquired deformity is determined by the clinician's evaluation of the limb abnormality and identification of the effector muscles. Muscles targeted for botulinum intervention to treat flexural deformities resulting from DOD can include the superficial digital flexor, biceps brachii, deep digital flexor (DDF), common digital extensor, flexor carpi ulnaris and radialis.

Congenital muscle contraction often involves the carpus and fetlock of both forelimbs and is suspected to result from intrauterine malposition, and/or maternal exposure to toxins or pathogens. Embertson, “Veterinary Clinics of North America” Equine Practice. Vol. 10, No. 2. (August 1994). Although poorly defined, foals with congenital contraction often exhibit excessive tone of the SDF and DDF muscles. Thus, the SDF and DDF can be targeted for botulinum intervention to treat congenital muscle contraction. Additional muscles targeted for botulinum intervention to treat congenital contraction can include flexor carpi radialis and flexor carpi ulnaris.

Rotational Deformities

A rotational deformity can be an abnormal axial rotation of an anatomical structure. Anatomical structures can be a neck, torso, limb, or pelvis. For a limb, a rotational deformity is an axial rotation of a limb segment relative to the segment proximal to it or the sagittal axis of the thorax. The deviation can be inward or outward, e.g., inward or outward rotation of the digit relative to the sagittal body plane.

While rotational deformities are of uncertain origin, current theory focuses on variation of bony growth plates and orthopedic abnormalities. However, while not wishing to be bound by a particular theory, the inventors believe that muscles act in an elastic manner, damping and modifying the spiral loading of the limbs' boney column during the protraction, stance or retraction phase of stride. Abnormal or unbalanced tone may exert excessive torsional forces on the skeleton, thereby causing or contributing to a rotational deviation. By decreasing the tension in one or more muscles, the excessive torsional forces can be alleviated, thereby reducing the degree of axial rotation and/or the severity of the rotational deformity.

Examples of such muscles that could be targeted for botulinum intervention in the treatment of a rotational deformity include the lateral digital extensor, the common digital extensor, extensor carpi radialis, abductor digiti longus as well as muscles of the shoulder girdle or synsarcosis including brachiocephalicus, omotransversarius, trapezius, rhomboideus, cutaneous trunci, serratus cervicis and thoracis, infraspinatus, ascending, transverse and descending pectoral muscles, and latissimus dorsi.

In one example of a rotational deformity, the common digital extensor and ulnaris lateralis originate on the lateral aspect of the limb and cross from lateral to a distal and dorsal antebrachial insertion exerting a stabilizing rotational torque on the distal limb. The muscles of the shoulder girdle and synsarcosis impact posture and dampen the torsional effect and maintain the limb in a structurally balanced orientation. Muscles that can be targeted to treat this type of rotational deformity include one or more of the following: lateral digital extensor, the common digital extensor, extensor carpi radialis, abductor digiti longus as well as muscles of the shoulder girdle or synsarcosis including brachiocephalicus, omotransversarius, trapezius, rhomboideus, cutaneous trunci, serratus cervicis and thoracis, infraspinatus, ascending, transverse and descending pectoral muscles, and latissimus dorsi.

In another example, increased tone of the pectoralis muscles can result in rotation of the humerus. This in turn results in a complex set of rotational forces transmitted to the distal phalanx. The forces of these muscles combined with antagonist muscle forces can create or exacerbate the rotational anomalies of the developing horse and likely vary by the specific muscles or nerve pathway involved in the dysfunction. Muscles that can be targeted for botulinum intervention to treat this type of rotational deformity include one or more of the following: lateral digital extensor, the common digital extensor, extensor carpi radialis, abductor digiti longus as well as muscles of the shoulder girdle or synsarcosis including brachiocephalicus, omotransversarius, trapezius, rhomboideus, cutaneous trunci, serratus cervicis and thoracis, infraspinatus, ascending, transverse and descending pectoral muscles, and latissimus dorsi. The methods according to the present invention are highly suitable for treating, preventing, or reducing the severity of flexural or rotational abnormalities, such as those described above, of a quadruped animal. A preferred embodiment of the invention relates to the treatment of flexural or rotational deformities, particularly in immature hooved or domesticated animals such as foals and fillies, with an effective amount of a botulinum toxin.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve reducing the degree of the deformity or alleviating one or more symptoms of the deformity, although not necessarily all of the symptoms, of the deformity, or attenuating the progression of the deformity. Eliminating the deformity from the animal is an optimal outcome of the practice of the methods of the invention. Embodiments of the invention include administering compositions of the invention to prevent or reduce the incidence or severity of musculoskeletal deformities, particularly flexural or rotational deformities.

A “quadruped animal” to be treated according to the present methods can refer to, e.g., Equidae (e.g., horse, ass, zebra), Bovidae (e.g., cattle, bison, sheep, goat, yak, impala, antelope, hartebeest, wildebeest, gnu, gazelle, water buffalo, duiker), Cervidae (e.g., deer, elk, moose, reindeer, pudu, bororo, brocket, guemal, muntjac), Suidae (e.g., pig, hog, boar), Canidae (domesticated dog, wolf, fox, coyote, jackel), Felidae (e.g., domesticated cat, cheetah, ocelot, lynx, bobcat, mountain lion, leopard, puma, lion, jaguar, tiger), Rodentia (e.g., mouse, rat, guinea pig, chinchilla, agouti, porcupine, beaver, gopher), Lagomorpha (e.g., rabbit, jackrabbit, hare, pika), Camelidae (e.g., camel, llama, alpaca, guanaco, vicugna), Ursidae (e.g., bear, panda), Procyonidae (e.g., raccoon, coati, olingo), Mustelidae (polecat, weasel, ferret, mink, fisher, badger, otter, wolverine, marten, sable, ermine), Elephantidae (e.g., elephant), rhinoceros, and hippopotamus. In some embodiments, the quadruped animal is a domestic or commercially used animal. In some embodiments, the quadruped animal is a horse.

The quadruped animal to be treated according to the present methods can be an immature animal or an animal still undergoing musculoskeletal growth. In some embodiments, the quadruped animal can be less than 3 years, 2 years, 1 year, 6 months, 5 months, 4, months, 3 months, 2 months, or 1 month of age. In some embodiments, the quadruped animal is a filly or a colt. In some embodiments, the quadruped animal is a foal.

According to the invention, a method of treating a musculoskeletal deformity of a distal limb in a quadruped animal comprises administering, such as by injection or intramuscular injection, an effective amount or a therapeutically effective amount of botulinum toxin to one or more target muscles in a limb (forelimb or hind limb) or shoulder of the animal. The treatment methods are directed to ameliorating, preventing, inhibiting, reversing, attenuating, alleviating, abrogating, minimizing, suppressing, reducing, decreasing, diminishing, stabilizing, eradicating, curing, or eliminating flexural or rotation deformities and/or their associated symptoms caused thereby. The musculoskeletal deformity can be acquired or congenital. The target muscle(s) within the limb or shoulder can vary depending on the nature of the musculoskeletal deformity.

In the case of an animal with a rotational deformity, at least one target muscle can be one that imparts torsional forces on one or more skeletal bones of the limb. In some embodiments treating a rotational deformity, the one or more target muscles can be selected from lateral digital extensor, the common digital extensor, serratus cervicis, serratus thoracis, infraspinatus, ascending pectoral muscles, descending pectoral muscles, brachiocephalicus, omotransversarius, trapezius, rhomboideus, cutaneous trunci, and latissimus dorsi.

In the case of an animal with a flexural deformity, the target muscle(s) can include one or more dorsal shoulder muscles such as brachiocephalicus, omotransversarius, trapezius, rhomboideus, cutaneous trunci, and latissimus dorsi). In addition to one or more dorsal shoulder muscles, the target muscles can further include deep flexor and/or superficial flexor muscles.

To identify the target muscles, a clinician can physically examine the quadruped animal to identify areas of abnormal or relatively higher muscle tension as compared to nearby muscles. For example, the clinician can conduct a tactile examination to identify the muscles with abnormal or relatively higher muscle tension. Electromyography and/or ultrasound may also be used for identification of muscles with increased tone.

The botulinum toxin is a neurotoxic protein that prevents the release of acetylcholine from the nerve terminal by disrupting the release mechanism of the acetylcholine-containing vesicles. Blocking the release of acetylcholine from the nerve terminal temporarily prevents the contraction of a muscle, thereby decreasing the tone of the muscle. Through such action, with methods described herein, the balance of forces on the limb or shoulder can be altered, thereby treating the flexural or rotational abnormality.

Botulinum toxin consists of seven distinct serotypes, A through G. The present invention should not be limited to any one serotype of the toxin. However, in a preferred embodiment, the botulinum toxin Type A is administered to the animal. Type A is commercially available and can be obtained as Botox, Allergan, Irvine, Calif.; Dysport, Ipsen, Paris, France; and Xeomin, Merz, Greensboro, N.C. Type B is also commercially available and can be obtained as Myobloc, Solstice Neurosciences, San Francisco, Calif.

Typically, the physiologic effect of botulinum toxin lasts 10-12 weeks. Whether multiple treatment sessions are required can depend on the degree of success of the previous treatment. In some embodiments, a quadruped animal will undergo a single treatment. In other embodiments, a quadruped can have 2, 3, 4, 5 6, 7, 8, 9, or 10 treatment sessions. In some embodiments, the time period between treatment sessions can be 1, 2, 3, 4, 5, 6, 7, 8, 9, 10, 11, 12, 13, 14, 15, or 16 weeks.

An effective amount of the botulinum toxin refers to the quantity (amount) that induces a desired response in the animal subject after administration. The total effective amount per limb can vary depending on the type of botulinum toxin, the brand of botulinum toxin, the size of the quadruped, and the size and involvement of the affected muscles in the limb. Optimally, an effective amount produces a therapeutic effect in the absence of, or with little or virtually no, adverse effects or toxicity in the animal. Alternatively, any adverse effects associated with an effective amount are optimally outweighed by the therapeutic benefit achieved. In one embodiment, the total effective amount intramuscularly injected per limb can be an amount equivalent to between about 1 U and about 600 U of Botox® botulinum toxin Type A, such as an amount equivalent to between about 20 U and about 400 U of Botox® botulinum toxin Type A per limb, between about 40 U to 200 U of Botox® botulinum toxin Type A per limb, or between about 50 U and 100 U of Botox® botulinum toxin Type A per limb.

Alternatively, the botulinum toxin, can be administered in an amount equivalent to between about 10⁻³ U and about 60 U of Botox® botulinum toxin type A per kg of the animal per limb to be treated. Preferably, the botulinum toxin used is administered in an amount equivalent to between about 10⁻² U and about 50 U of Botox® botulinum toxin type A per kg per limb to be treated. More preferably, the botulinum toxin is administered in an amount equivalent to between about 10⁻¹ U and about 40 U of Botox® botulinum toxin type A per kg per limb to be treated. Most preferably, the botulinum toxin is administered in an amount of between about 1 U of Botox® botulinum toxin type A per kg and about 30 U/kg per limb to be treated. In a particularly preferred embodiment of the present disclosed methods, the botulinum toxin is administered in an amount equivalent to between about 1 U and about 20 U of Botox® botulinum toxin type A perkg per limb to be treated. It is understood that other brands and/or serotypes of botulinum toxin can have different potency and the dosage can be adjusted accordingly.

As will be appreciated by those having skill in the art, the specific dose can be calculated in the light of the relevant circumstances, including the approximate weight or volume of the muscle targeted for injection, the severity of the tension of the targeted muscle, the age of the animal, and response of the individual animal receiving treatment. Exact dosages can be determined based on standard dose-response studies. Therapeutically effective doses for treatment of afflicted animals can be determined by titrating the amount of the active product given to the animal to arrive at the desired therapeutic effect, while minimizing side effects.

The composition effective for treating flexural and rotational deformities according to the invention comprises botulinum toxin. More particularly, the composition comprises botulinum toxin, e.g., type A, and a pharmaceutical carrier suitable for injection. Typically, the botulinum toxin is administered after being reconstituted according to the manufacturer's recommendations at a desired dilution. In one example, a quantity of 100 U of powdered Botox® is dissolved in 2-4 mL of preservative free, sterile saline. The region to be injected is prepped in a sterile fashion. The target muscle is first identified anatomically; the muscle can then be accessed utilizing, for example, an Ambu Neuroline Inoject 24 gauge, 2-3 inch coated needle electrode using EMG guidance. Botulinum toxin is then injected through this needle into one or more sites in the target muscle. For example, botulinum toxin can be injected into 1, 2, 3, 4, 5, 6, 7, 8, 9, 10, or more sites in the target muscle. The injection sites can be spaced apart across the length and width of the muscle. In some embodiments, the spacing between injection sites for a target muscle can be 1, 2, 3, 4, or 5 inches. The dosage per injection site can be an amount equivalent to 0.5 U, 1 U, 2 U, 3 U, 4 U, 5 U, 6 U, 7 U, 8 U, 9 U, or 10 U of Botox® botulinum toxin type A. In some embodiments, a target muscle can have 2 to 6 injection sites and the dosage per injection can be an amount equivalent to 2 U to 8 U of Botox® botulinum toxin type A. In a treatment cycle, a single muscle can be targeted or a plurality of muscles, e.g., 2, 3, 4, 5, or 6 muscles) can be targeted for botulinum intervention. The total dosage is the total amount that is injected per limb in a treatment session.

The methods of the invention further embrace the administration of pharmaceutically acceptable formulations of the botulinum toxin composition either alone or in combination with other agents or therapies. For example, physical therapy may be utilized in combination with the administration of botulinum toxin. Other therapies which have demonstrated benefit and suitable for use with botulinum include hoof farriery, swimming and control of exercise.

### EXAMPLES

### Example 1—Flexural Deformity

A 3 month old walking horse colt that had developed an increased pastern angle bilaterally. The colt was otherwise healthy with no other evidence of leg disease or discomfort. He was beginning to exhibit changes in shape of his hoof including the development of a more cylindrical hoof.

50 U of Botox® botulinum toxin type A was divided into the bilateral superficial digital flexor muscles. 12.5 U aliquots were administered through 4 injections at 4 sites on each side.

The horse tolerated the procedure well with no adverse effects noted. The horse was monitored daily showing no signs of adverse effects. The pastern angle stabilized and the horse remains functional with steep pastern angles and low heels.

### Example 2—Flexural Deformity

A one-year old thoroughbred colt exhibited normal orthopedic development until approximately 6 months of age when he exhibited a steepening of the pastern angle associated with posterior displacement of the knee. Examination showed no other abnormalities.

The horse received a total of 200 U of Botox® botulinum toxin type A administered in 25 unit aliquots divided evenly between the bilateral deep digital flexor muscles (DDF). The horse tolerated the procedure well with no adverse effects noted. At 2 weeks post injection, no further deterioration was observed. The horse entered race training and has performed in normal duties.

### Example 3—Flexural Deformity

A four-month old thoroughbred filly developed an increasing pastern angle, increased hoof angle, and “back in the knee” unilaterally. The horse was otherwise healthy with no evidence of underlying hoof or leg disease.

The filly received 75 U of Botox® botulinum toxin type A in the DDF. This was administered in 25 U aliquots at 3 injection sites in the DDF. The horse tolerated the procedure well with no adverse effects noted. At two weeks post injection the filly showed no deterioration in orthopedic alignment and no gait abnormalities. At 8 months of age the angles had returned to a normal conformation. The filly is currently in race training.

### Example 4—Rotational Deformity

This 3-month-old thoroughbred colt developed an inward rotation of the left hoof at approximately 2 months of age. The inward rotation exhibited slow progression and so intervention commenced. At 5 months of age, the colt received 100 U Botox® botulinum toxin type A into the common digital extensor (75 U) and the lateral digital extensor (25 U). The horse tolerated the procedure well. At two weeks post injection, the colt was stable showing no evidence of gait abnormality or deterioration in orthopedic alignment. Colt was race training as a 2 year old.

### Example 5—Rotational Deformity

This thoroughbred filly developed internal rotation anomalies of her right foot at approximately 6 months of age. Examination showed no etiology.

She received 100 U of Botox® botulinum toxin type A divided by 10 U aliquots and administered into the right common digital extensor and the right lateral digital extensor, 5 injection sites each. The horse tolerated the procedure well. Two weeks post injection the filly showed no evidence of adverse gait changes. At 1 year of age, the horse was stable with no further deterioration of the rotational abnormalities. At 3 years of age, the horse has a normal conformation.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

1. A method for treating a rotational abnormality in a limb of a quadruped animal comprising:
injecting a Botulinum Toxin, in a therapeutically effective amount, into one or more muscles of the limb or shoulder of the animal.

2. The method of claim 1, wherein the Botulinum Toxin is injected into at least one muscle that imparts torsional forces on one or more skeletal bones of the limb.

3. The method of claim 1, wherein the Botulinum Toxin is injected into at least one muscle that crosses a midline of a torso, pelvis, or neck to exert torsional forces on one or more skeletal bones of the torso, pelvis, or neck.

4. The method of claim 1, wherein the one or more muscles of the limb or shoulder are selected from lateral digital extensor, the common digital extensor, extensor carpi radialis, abductgor digiti longus, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi.

5. A method for treating a musculoskeletal deformity of a quadruped animal comprising:
injecting a Botulinum Toxin, in a therapeutically effective amount, into at least one muscle that exerts torsional forces on one or more skeletal bones of the anatomical structure.

6. The method of claim 5, wherein the one or more muscles that impart rotational forces on an anatomical structure are selected from lateral digital extensor, the common digital extensor, extensor carpi radialis, abductgor digiti longus, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi.

7. The method of claim 1, wherein the deformity is a rotational deformity.

8. The method of claim 5, wherein the musculoskeletal deformity is in a limb of the quadruped animal and the method comprises
injecting a Botulinum Toxin, in a therapeutically effective amount, into
1) a superficial or deep digital flexor muscle of the distal limb and
2) one or more muscles selected from a common digital extensor, a right lateral digital extensor, ulnaris lateralis, flexor carpi ulnaris, lacertis fibrosis, flexor carpi radialis, extensor carpi radialis, biceps brachii, triceps, serratus ventralis, serratus cervicis, serratus thoracis, infraspinatus, brachiocephalicus, omotransversarius, trapezius, rhomboideus, cutaneous trunci, ascending pectoral muscles, descending pectoral muscles, transverse pectoral muscles, and latissimus dorsi.

9. A method of treating a flexural or rotational deformity of a limb of a quadruped animal comprising:
injecting a Botulinum Toxin, in a therapeutically effective amount, into one or more muscles of the distal limb selected from lateral digital extensor, the common digital extensor, ulnaris lateralis, flexor carpi ulnaris, lacertis fibrosis, flexor carpi radialis, extensor carpi radialis, biceps brachii, triceps, serratus ventralis, serratus cervicis, serratus thoracis, infraspinatus, ascending pectoral muscles, descending pectoral muscles, transverse pectoral muscles, brachiocephalicus, omotransversarius, trapezius, rhomboideus, cutaneous trunci, and latissimus dorsi

10. The method of claim 8, wherein the deformity is a rotational deformity.

11. (canceled)

12. (canceled)

13. The method of claim 1, wherein the botulinum toxin is injected into one or more muscles of a forelimb synsarcosis, wherein the one or more muscles of the forelimb synsarcosis are selected from brachiocephalicus, omotransversarius, trapezius, rhomboideus, cutaneous trunci, ascending pectoral muscles, descending pectoral muscles, transverse pectoral muscles, and latissimus dorsi.

14. The method of claim 13, wherein at least 30% of a total dosage per limb of the botulinum toxin is injected into the one or more muscles of the forelimb synsarcosis.

15. The method of claim 1, wherein the total dosage per limb of the Botulinum Toxin is an amount equivalent to about 40 Units to 200 Units of Botox®.

16. (canceled)

17. The method of claim 1, wherein the quadruped animal is a filly or a colt or less than 3 years old.

18. The method of claim 1, wherein the quadruped animal is a foal or less than a year old.

19. (canceled)

20. The method of claim 1, wherein the deformity is acquired or congenital.

21. (canceled)

22. The method of claim 9, wherein the deformity is a flexural deformity and is caused in part from contracture of the deep digital flexor tendon, contracture of the superficial digital flexor tendon, contracture of the flexor carpi ulnaris flexor, carpi radialis, brachialis, ulnaris lateralis.

23. The method of claim 1, wherein the rotational abnormality is caused in part from contracture of one or more of the following: the pectoralis muscle, subscapularis, deltoideus, serratus ventralis, trapezius, rhomboideus, brachiocephalicus, latissimus dorsi, teres major, superspinatus, coracobrachialis, common digital extensor, lateral digital extensor, and abductor digiti longus.

24. The method of claim 9, wherein the quadruped animal is a hooved animal and exhibits an abnormally low pastern angle or an abnormally high pastern angle.

25. (canceled)
